(19) United States
(12) Patent Application Publication
(10) Pub. No.: US 2014/0046342 A1
(43) Pub. Date: Feb. 13, 2014

(54) METHOD AND SYSTEM FOR AIDING IN THE POSITIONING OF A MEDICAL INSTRUMENT ON THE HEAD OF A PATIENT

(71) Applicants: Pierre Hellier, Thorigne-Fouillard (FR); Xavier Morandi, Rennes (FR); Benoît Combes, Vitre (FR); Charles Garraud, Rennes (FR); Sylvain Prima, Rennes (FR)

(72) Inventors: Pierre Hellier, Thorigne-Fouillard (FR); Xavier Morandi, Rennes (FR); Benoît Combes, Vitre (FR); Charles Garraud, Rennes (FR); Sylvain Prima, Rennes (FR)

(73) Assignees: INRIA INSTITUT NATIONAL DE RECHERCHE EN INFORMATIQUE ET EN AUTOMATIQUE, LE CHESNAY (FR); CENTRE HOSPITALIER UNIVERSITAIRE DE RENNES, RENNES CEDEX (FR); UNIVERSITE DE RENNES 1, RENNES CEDEX (FR)

(21) Appl. No.: 13/981,934

(22) PCT Filed: Jan. 25, 2012
(86) PCT No.: PCT/EP2012/051119

§ 371 Date: Oct. 14, 2013

(30) Foreign Application Priority Data
Jan. 26, 2011 (FR) 1150606

(51) Int. Cl. A61B 19/00

(52) U.S. Cl. CPC A61B 19/201
USPC 606/130

(57) ABSTRACT

A system for aiding in the positioning of a medical instrument on the head of a patient, characterized in that it includes a means for predicting the location of a target intracranial area of the medical instrument, comprising a generic three-dimensional model of a head comprising a scalp and a generic target intracranial area, a three-dimensional working image of the scalp of the head of the patient, surface registration means suitable for establishing a registration transform from the scalp of the generic model toward the working image of the scalp of the head of the patient, a means for resampling the generic target intracranial area into a converted target area using with the registration transform, and a means for locating the target intracranial area of the patient from the locating of the converted target area, thereby enabling the positioning of the medical instrument on the head of the patient.

### CROSS-REFERENCE TO RELATED APPLICATIONS

This application is the National Stage of International Application No. PCT/EP2012/051119, filed Jan. 25, 2012. The International Application claims priority to French Application No. 11 50606, filed Jan. 26, 2011. The International Application published on Aug. 2, 2012 as WO 2012/101160. All of the above applications are incorporated herein by reference.

### FIELD OF THE INVENTION

The present invention relates to a system for aiding in the positioning of a medical instrument on the head of a subject and a method for aiding in the positioning associated therewith. The subject is an analysis subject or a patient.

### BACKGROUND

The invention applies to the field of transcranial magnetic stimulation (TMS). Transcranial magnetic stimulation is a cortical stimulation technique, the principle of which is based on generating a high-intensity focused magnetic field. This technique makes it possible to stimulate a target neuroanatomical area in a painless and noninvasive manner. The stimulation is done using a coil in which circulates a strong electrical current that causes the generation of a focused magnetic field. Due to the focal nature, the applications to neurology and psychiatry are numerous, for example the treatment of depression, etc.

To ensure the precision and reproducibility of the stimulation between treatment sessions, it is necessary and now traditional to use neuronavigation to guide the positioning of the stimulation coil on the patient's head.

Neuronavigation allows geometric matching of the actual anatomy of the patient with the patient's anatomy represented using an imaging method, preferably of the magnetic resonance imaging (MRI) type. Thus, it is possible to guide the clinician precisely toward the target area, located on the MRI, through real-time monitoring of the position of a stimulated point, and to compare it with the desired stimulation position.

Devices for aiding in the location of the anatomical cortical target are already known in the state of the art.

This includes document WO 2010/084,262, which describes a device making it possible to automatically match the patient's head with images of the patient acquired by MRI using a registration algorithm which, from the anatomical match points between the patient's head and the MRI images, manages to calculate the transformation of the three-dimensional reference of the patient to the reference of the MRI images.

Nevertheless, this type of guiding and the traditional neuronavigation techniques require the acquisition of an MRI image of the patient.

However, this imaging method is restrictive for cost and ease-of-access reasons.

In fact, this imaging being a reference exam for cerebral anatomy, the average waiting time in France was 34.6 days for this type of exam in January 2010. Furthermore, the average cost is [symbol] 300, which seems high in light of the large number of patients who can benefit from TMS for depression (depression is a major public health issue, with a prevalence rate of 15% of the population).

The invention aims to propose a system for aiding in the positioning of a medical instrument on the head of a patient making it possible to do away with images acquired by MRI while ensuring reliable positioning of the medical instrument.

To that end, the invention relates to a system for aiding in the positioning of a medical instrument of the aforementioned type, characterized in that it comprises means for predicting the location of a target intracranial area of the medical instrument, including a generic three-dimensional model of the head comprising scalp and a generic target intracranial area, a three-dimensional working image of the scalp of the patient's head, surface registration means suitable for establishing a registration transform from the scalp of the generic model toward the working image of the scalp of the patient's head, a means for resampling the generic target intracranial area into a converted target area using the registration transform, and a means for locating the target intracranial area of the patient from the locating of the converted target area, thereby enabling the positioning of the medical instrument on the patient's head.

According to specific examples, the system for aiding in the positioning has one or more of the following features, considered alone or in combination:

the three-dimensional working image of the scalp of the patient's head is an image of the x-ray type;
the three-dimensional working image of the patient's head is a surface image acquired using a three-dimensional location system;
the medical instrument is a coil of an intracranial magnetic stimulation device; and
the intracranial target area is the dorsolateral prefrontal cortex (DLPFC).

According to another aspect, the invention also relates to a method for aiding in the positioning of a medical instrument on the patient's head, characterized in that it includes the following steps

locating a generic target intracranial area on a generic three-dimensional model of the head comprising a scalp,
extracting the surface of the scalp of the head from the generic three-dimensional model,
extracting the surface of the scalp of the patient's head from a three-dimensional working image of the scalp of the patient's head,
surface registration suitable for establishing a registration transform of the scalp of the generic model to the working image of the scalp of the patient's head,
resampling the target generic intracranial area into a converted target area using the registration transform, and
locating the target intracranial area of the patient from the location of the converted target area making it possible to position the medical instrument on the patient's head.

According to one example, the method for aiding in the positioning has no step for acquiring images at least the patient's head by magnetic resonance imaging.

### BRIEF DESCRIPTION OF THE FIGURES

The invention will be better understood upon reading the following description, provided solely as an example and done in reference to the drawings, in which:

FIG. 1 is a diagrammatic view of a system for aiding in the positioning of the medical instrument on the patient's head according to the invention, and

FIG. 2 is an overview diagram illustrating the method implemented by the system for aiding in the positioning of FIG. 1.

### DETAILED DESCRIPTION

The invention will now be described in detail in reference to a specific cerebral neuroanatomical area: the dorsolateral prefrontal cortex (DLPFC). Roughly speaking, this neuroanatomical area corresponds to the interface between areas 9 and 46 of Brodmann's cytoarchitectonic map. However, the invention is in no way limited to that area, but rather applies to any cerebral area.

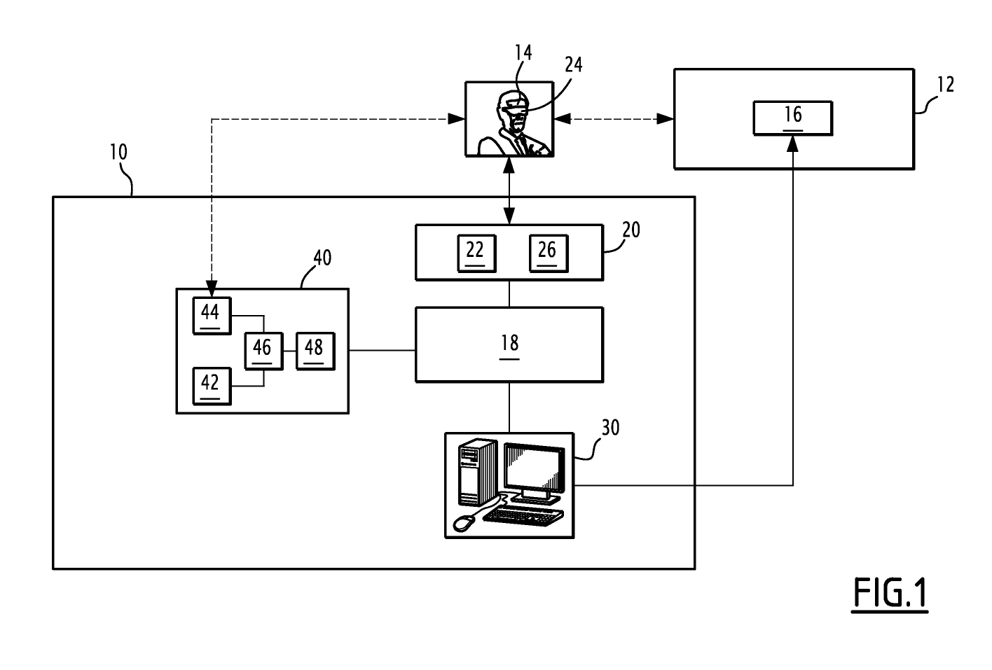

The aid system 10 illustrated in FIG. 1 is designed to position a medical instrument 12 on the head of the patient 14. The patient 14 is an analysis subject, for example an individual suffering from depression, obsessive-compulsive disorder, neuropathic pain or auditory schizophrenic hallucinations, and requiring transcranial magnetic stimulation of the dorsolateral prefrontal cortex of his brain.

Furthermore, the medical instrument 12 is preferably an electromagnetic stimulation device, for example for transcranial magnetic stimulations, having a stimulating coil 16, in which a strong electrical current passes that causes the generation of a focused magnetic field on the cortical target to be reached: the DLPFC.

The aid system 10 includes a neuronavigation device 18, i.e., a guiding device using images, and a positioning system 20, in three dimensions, so as to monitor the position of the point stimulated by the stimulation coil 16 in real time and compare it with the desired stimulation position.

The positioning system 20 has a positioning tool 22, such as a band 24 fastened around the head of the analysis subject 14, and a camera 26 directly or indirectly related to the positioning instrument 22. The camera 26 is for example a binocular camera. The band 24 is marked and can be recognized by the camera 26, and thus defines a reference point fixedly attached to the head of the subject 14.

Furthermore, the aid system 10 includes a computer device 40 having a first memory 42 capable of storing data, for example a memory of the random access memory (RAM) type.

This first memory 42 is arranged to store a generic three-dimensional map of at least part of the head of a model and at least the model brain, also called a map. The brain to which reference is made at this stage is a brain that can be qualified as a model brain or a generic brain. For example, it is a digitized MRI map of a model brain.

The generic three-dimensional mapping comprises the location of the target area, here the dorsolateral prefrontal cortex (DLPFC) and the scalp of the model head comprising the model brain.

The first memory 42 also stores data for precise designation of the target area: the DLPFC.

Furthermore, the computer device 40 includes a second memory 44 capable of storing data, for example of the RAM type. The second memory 44 is arranged to receive and store a working image of at least part of the head of the analysis subject.

The working image is a three-dimensional surface image of the scalp of the subject's head. It is obtained either using a medical imaging method other than magnetic resonance imaging (MRI), or by a location/three-dimensional tracking device (or optical tracking), for example the MICRONTRACKER device by the company CLARON TECHNOLOGY or the POLARIS® device by the company Northern Digital Inc. (NDI). Such a system comprises a binocular camera that films the scene, a marked band recognized by the camera and serving as a reference point fixedly attached with respect to the subject's head, a marked pointer recognized by the camera, and a computer connected to the camera. The binocular camera and the band of the three-dimensional location device are for example those of the positioning system 20.

The computer device 40 also includes surface registration means 46 that receives generic three-dimensional mapping data and working data from the first memory 42 and the second memory 44, respectively. It is from this data that the surface registration means 46 establishes a registration transform of the scalp of the generic model toward the working image of the scalp of the subject's head.

The computer device 40 also comprises a resampling means 48 for the target generic intracranial area into a converted target area according to the registration transformation transmitted by the surface registration means 46.

The resampling means 48 is suitable for transmitting the data representative of the converted target area to the neuronavigation device 18.

The aid device 10 comprises a man-machine interface 30 connected to the neuronavigation device 18 and capable of displaying viewing images transmitted by the neuronavigation device 18.

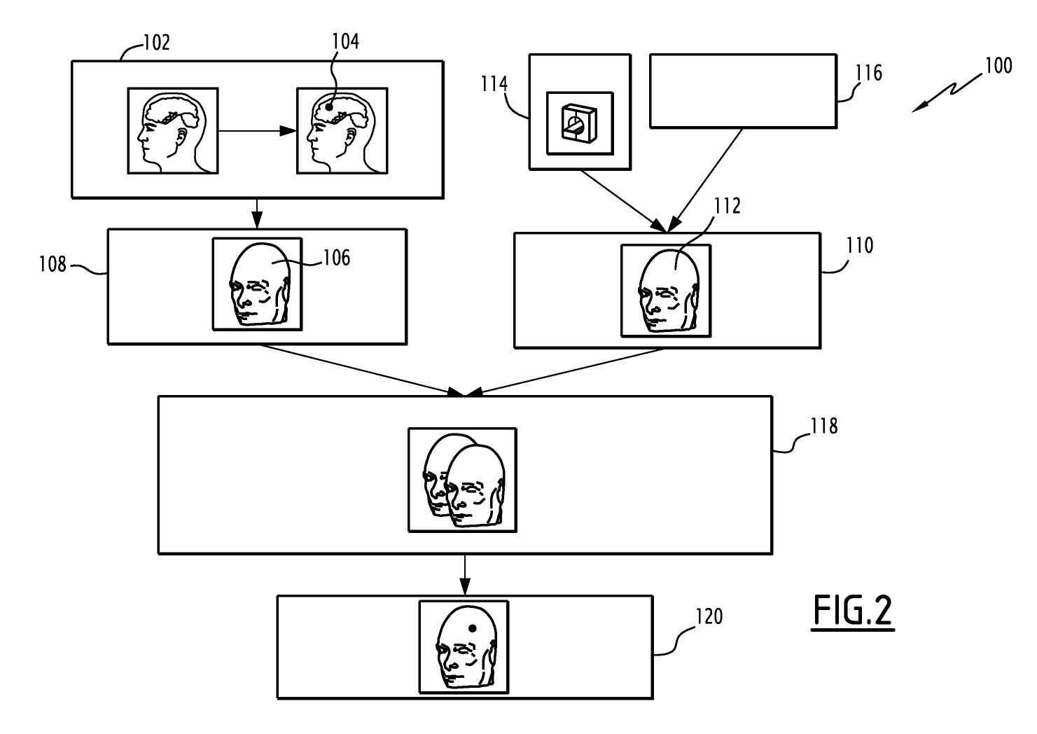

During operation, the aid system 10 carries out the steps of the method 100 illustrated in FIG. 2 for aiding in the positioning of a medical instrument on the patient's head.

During a step 102, a neuroanatomist locates a predefined target area 104, for example the left DLPFC, as well as anatomical reference points, which will then serve for the registration, on a whole-head digital MRI map, i.e., comprising a generic three-dimensional map of at least part of the head and the brain. This step is carried out once for all stimulation sessions.

For example, the whole-head digital MRI map is the MRI map described in the article by Lalys F, Haegelen C, Ferre J C, EI-Ganaoui O, Jannin P., entitled “Construction and assessment of a 3-T MRI brain template”, published in Neuroimage in August 2009.

Then, the surface 106 of the scalp of the map is extracted during a step 108 using traditional segmentation techniques.

The digital data corresponding to the extracted surface 106 of the scalp of the map are stored in the first memory 42 of the computer device 40 of the aid system 10.

In parallel, during a step 110, the surface 112 of the patient's scalp is extracted.

To that end, if medical imaging other than an MRI is done beforehand on the patient's head during a step 114, for example x-ray scanner imaging (or CT for “Computed Tomography”), the surface 112 of the scalp is automatically extracted from the x-ray scanner images using a traditional segmentation technique by the computer device 40.

According to an alternative embodiment, if no medical imaging method has been applied beforehand on the patient, for example neither magnetic resonance imaging (MRI) nor x-ray scanner (CT), the step 110 for extracting the patient's scalp requires a step 116 for acquiring and reconstructing the patient's scalp carried out by a three-dimensional location system.

The patient is situated in the field of vision of the camera 26 of the three-dimensional location system. The band 24 of that three-dimensional location system is fastened on the head of the patient and recognized by the camera 26. The band then defines a reference point fixedly attached with respect to the patient's head.

The operator then uses a pointer of the three-dimensional location system also recognized by the camera 26 and thus makes it possible to sample the patient's head with a cloud of points defined by the position of the pointer in the reference point defined by the band 24. The camera films the scene, i.e., the different positions of the pointer.

The signals corresponding to the points are sent to the computer device 40 and are stored in a memory of the computer device 40. They are digitized so as to be processed by the computer device 40 to extract or reconstruct the surface 112 of the scalp therefrom using that cloud of points. The recorded cloud of points is preprocessed by smoothing, then the aberrant data (or points) are excluded and normals to the curves defined by the cloud of points are calculated. Lastly, a Poisson algorithm makes it possible to reconstruct a smooth and regular surface from the sparse cloud of points.

The surface 112 of the patient's scalp or any other digital data representative thereof is then recorded in the second memory 44 of the computer device 40.

The precision of the Poisson surface reconstruction has been validated from a ghost, for example a mannequin, having undergone sampling using the method described above and x-ray scanner imaging. It has been evaluated by performing a surface registration, using an algorithm of the iterative closest point (ICP, “Iterative Closest Point”) algorithm type, from the cloud of points toward the surface of the scalp extracted from the x-ray scanner and applying the calculated transformation to the reconstructed Poisson surface from the cloud of points.

The results of the evaluation show:
a mean point-surface error of 1.5 mm between the initial cloud of points and the reconstruction of the scalp from the image obtained by x-ray scanner, and
a mean point-surface error of 1.6 mm between the Poisson reconstruction and the reconstruction of the scalp from the image obtained by x-ray scanner.

The method 100 continues with a step 118 for registration the surface of the scalp of the map and the surface of the patient's scalp. In fact, the surface of the scalp of the map is deformed on the surface of the patient's scalp.

This surface registration step 118 is applied by the surface registration means 46 of the computer device 40 in several steps applied successively.

The surface registration means 46 receives the generic three-dimensional mapping data, i.e., the surface of the scalp of the model, and the working data, i.e., the surface of the scalp of the patient, from the first memory 42 and the second memory 44, respectively.

First, the surface registration means 46 carries out an initialization step of the registration from the anatomical reference points located on the map by the neuroanatomist during the step 102 and on the patient during step 110 for extracting the patient's scalp either from images acquired by x-ray scanner in 114, or from the sampling of the patient's scalp in 116.

Next, the surface registration means 46 applies a rigid surface registration of the reconstruction of the extracted scalp from the MRI map toward the surface of the patient's scalp using an algorithm of the ICP type.

Then, he performs an affine surface registration of the construction of the scalp extracted from the MRI map toward the surface of the scalp of the patient, using an algorithm of the ICP type.

Next, the surface registration means 46 performs a nonlinear surface registration of the reconstruction of the scalp extracted from the MRI map toward the surface of the patient's scalp. For example, such a nonlinear surface registration is described in the article by Benoît Combès and Sylvain Prima, entitled “An efficient EM-ICP algorithm for symmetric consistent non-linear registration of point sets” and presented during the 13th international conference “Medical Image Computing and Computer-Assisted Intervention (MICCAI)”in September 2010.

During a step 120, the registration transform calculated during the surface registration step 118 is applied to the left DLPFC located by the neuroanatomist on the MRI map to determine its position with respect to the surface of the scalp extracted during step 110. Of course, the registration transformation includes all of the transformations calculated during the different registration steps described above.

One thus obtains the coordinates of the left DLPFC in the anatomical reference point of the subject represented by the reconstruction of the scalp, which are transmitted to the neuronavigation device. The latter then matches the actual reference point in which the analysis subject evolves and the anatomical reference point of the subject (the surface of the scalp).

A viewing image is formed from viewing data transmitted by the neuronavigation device and which match, at least partially, the working image and the converted map, while indicating an area in the viewing image that corresponds to the converted designation data.

This viewing image is transmitted to the user interface 30, which displays it so that it can be viewed by the clinician or the operator.

Thus, the left DLPFC is located in the patient automatically, without any particular intervention by clinician.

The operator uses the viewing image to position a coil 16 for the emission of electromagnetic pulses. The positioning of the coil of the TMS device is adjusted with respect to the viewing images presented on the user interface 30, by means of the positioning tool 22 and the camera 26.

Subsequently, during transcranial magnetic stimulation (TMS) sessions, the system for aiding in the cerebral location according to the invention allows precise tracking, in real-time, of the area actually stimulated by the magnetic stimulations of the TMS using the neuronavigation device 18.

Validation

A validation of the methodology for the scalp surface registration from the map/scalp of the patient to locate the left DLPFC was carried out on a group of several patients:
a group A of analysis subjects having undergone x-ray scanner imaging in addition to magnetic resonance imaging, and
a group B of analysis subjects with no imaging method other than their image obtained by magnetic resonance imaging (MRI).

The left DLPFC was first identified by a neuroanatomist on each patient MRI and on the Jannin MRI map, the complete reference for which is indicated above.

Then, the scalp from the MRI map was segmented traditionally.

Next, for each subject, the following method was applied:
A. Extraction of the surface of the patient's scalp:
   For group A, using a traditional segmentation technique of the scalp from the images obtained by x-ray scanner.
   For group B, according to step 110 for reconstructing the scalp without an imaging method described above using a three-dimensional location device.
B. Map—patient surface registration according to step 118 for scalp surface registration from the MRI map—patient's scalp described above.
   B.1. Rigid registration of the surface of the patient's scalp extracted toward the MRI of the patient:
      For group A, by rigid volume registration of the x-ray scanner image of the subject and his MRI image.
      For group B, rigid surface registration of the cloud of points obtained in the preceding step and the surface of the scalp extracted from the MRI.
   B.2. Direct volume registration of the MRI from the map toward the MRI of the patient using the method for aiding in Cerebral Location described in document WO 2010/084,262.
C. Application of transformations calculated during step B to the target area (left DLPFC) pointed to on the map by the neuroanatomist.
D. Comparison of the position of the DLPFC identified by the neuroanatomist on the MRI of the patient, the DLPFC reset using the method according to the invention and the DLPFC reset using the volume method described in document WO 2010/084,262.

Results

For group A, the obtained results are:
a mean error of 9.65 mm between the location of the DLPFC calculated from the surface registration and the location of the DLPFC identified in the MRI of the patient by the neuroanatomist, and
a mean error of 12.1 mm between the location of the DLPFC calculated from the surface registration and the location of the DLPFC calculated from the volume registration.

For group B, the obtained results are:
a mean error of 8.9 mm between the location of the DLPFC calculated from the surface registration and the location of the DLPFC identified on the MRI of the patient by the neuroanatomist, and
a mean error of 8.7 mm between the location of the DLPFC calculated from the surface registration and the location of the DLPFC calculated from the volume registration.

It will thus be understood that such systems and method according to the invention for aiding in the positioning of an instrument on a patient's head from an anatomical map make it possible to ensure reliable positioning of the instrument and consequently excellent reproducibility between stimulation sessions, from knowledge of the surface morphology of the patient.

Furthermore, the system allows easy and quick neuronavigation on a map, since it does not require MRI imaging. Furthermore, the system and method are less expensive than those of the prior art, since the cost of the MRI exam is eliminated.

The invention has been described in the context of the stimulation of the dorsolateral prefrontal cortex (DLPFC). Of course, the invention is in no way limited to this area, but on the contrary applies to any cerebral area, for example the motor cortex.

1. A system for aiding in the positioning of a medical instrument on the head of a subject, having a unit predicting the location of a target intracranial area of the medical instrument, comprising:
a generic three-dimensional model of the head comprising scalp and a generic target intracranial area,
a three-dimensional working image of the scalp of the patient's head,
surface registration device suitable for establishing a registration transform from the scalp of the generic model toward the working image of the scalp of the patient's head,
a resampler resampling the generic target intracranial area into a converted target area using the registration transform, and
a locator locating the target intracranial area of the patient from the locating of the converted target area, thereby enabling the positioning of the medical instrument on the patient's head.

2. The system for aiding in the positioning of a medical instrument according to claim 1, wherein the three-dimensional working image of the scalp of the patient's head is an image of the x-ray type.

3. The system for aiding in the positioning of a medical instrument according to claim 1, wherein the three-dimensional working image of the patient's head is a surface image acquired using a three-dimensional location system.

4. The system for aiding in the positioning of a medical instrument according to claim 1, wherein the medical instrument is a coil of an intracranial magnetic stimulation device.

5. The system for aiding in the positioning of a medical instrument according to claim 1, wherein the intracranial target area is the dorsolateral prefrontal cortex (DLPFC).

6. A method for aiding in the positioning of a medical instrument on the patient's head, comprising the steps of:
locating a generic target intracranial area on a generic three-dimensional model of the head comprising a scalp,
extracting the surface of the scalp of the head from the generic three-dimensional model,
extracting the surface of the scalp of the patient's head from a three-dimensional working image of the scalp of the patient's head,
surface registration suitable for establishing a registration transform of the scalp of the generic model to the working image of the scalp of the patient's head,
resampling the target generic intracranial area into a converted target area using the registration transform, and
locating the target intracranial area of the patient from the location of the converted target area making it possible to position the medical instrument on the patient's head.

7. The method for aiding in the positioning of a medical instrument on a patient's head according to claim 6, wherein no step for acquiring images at least the patient's head by magnetic resonance imaging is applied.
